(19) United States
(12) Patent Application Publication
(10) Pub. No.: US 2020/0187898 A1
(43) Pub. Date: Jun. 18, 2020

(54) A LAPAROSCOPIC ADAPTER, AN ECHOCARDIOGRAPHY PROBE AND A METHOD FOR COUPLING THE ADAPTER TO THE PROBE

(71) Applicant: KONINKLIJKE PHILIPS N.V., EINDHOVEN (NL)

(72) Inventors: Grzegorz Andrzej TOPOREK, BOSTON, MA (US); Aleksandra POPOVIC, BOSTON, MA (US)

(21) Appl. No.: 16/628,314

(22) PCT Filed: Jul. 5, 2018
(86) PCT No.: PCT/EP2018/068266

§ 371 Date: Jan. 3, 2020

Related U.S. Application Data
(60) Provisional application No. 62/529,654, filed on Jul. 7, 2017.

(51) Int. Cl. A61B 8/12; A61B 8/00; A61B 8/08; A61B 46/10

(52) U.S. Cl. CPC A61B 8/12; A61B 8/445; A61B 8/467; A61B 1/3132; A61B 8/483; A61B 8/4466; A61B 46/10; A61B 8/0883

(57) ABSTRACT

A 3D echocardiography probe (20) (e.g., a 3D transesophageal echocardiography probe or a 3D intracardiac echocardiography probe) can be adapted into a 3D laparoscopic ultrasound probe (10a) for laparoscopic procedures. The 3D echocardiography probe includes a flexible shaft (22d, 22p) to which a laparoscopic adapter (30a) can be coupled. The laparoscopic adapter comprises a laparoscopic sleeve (31) configured to partially encircle a portion of the flexible shaft of the 3D echocardiography probe and a probe handle (33a) mountable to the laparoscopic sleeve (31).

### FIELD OF THE INVENTION

The present disclosure generally relates to laparoscopic procedures (e.g., prostatectomy, aplenectomy, nephrectomy and hepatectomy). The present disclosure specifically relates to an adaption of a three-dimensional (“3D”) echocardiography probe into a 3D laparoscopic ultrasound (“LUS”) probe for the performance of a laparoscopic procedure.

### BACKGROUND OF THE INVENTION

During minimally invasive laparoscopic procedures, any endoscopic feedback provided to a surgeon has proved to be limited. Specifically, the surgeon using endoscopic camera may only view an outer surface of an organ. Therefore surgeons lose their tactile feedback, sense of orientation and all other information that are usually provided during conventional surgery. To overcome this endoscopic limitation, two-dimensional (“2D”) laparoscopic ultrasound (“LUS”) probes designed to have direct contact with a parenchyma of an organ were introduced to provide an intra-operative visualization of the inner structures of the organ. A clear disadvantage of 2D ultrasound is lack of volumetric information which could be of interest for precise assessment of the region of interest of the organ in relation to adjacent critical structures. Additionally, since laparoscopic procedures use a fixed fulcrum as an access point, the inverted hand-instrument movement make it very difficult to understand a location and an orientation of an ultrasound image plane with respect to the organ. Thus, limiting a field of view of 2D LUS probes to a single plane may lead to a misdiagnosis and/or ineffective treatment of the organ.

### SUMMARY OF THE INVENTION

Currently, while 3D ultrasound probes exist, 3D ultrasound probes for use during laparoscopic procedures do not exist. The present disclosure describes an adaption of a 3D echocardiography probe into a 3D laparoscopic ultrasound (“LUS”) probe.

For purposes of describing and claiming the inventions of the present disclosure:

(1) the term “laparoscopic procedure” broadly encompasses any and all types of laparoscopic procedures, as known in the art of the present disclosure or hereinafter conceived, for an imaging, a diagnosis and/or a treatment of a patient anatomy;

(2) the term “3D echocardiography probe” broadly encompasses any and all types of probes, as known in the art of the present disclosure or hereinafter conceived, incorporating an ultrasound transducer integrated within a flexible shaft for a ultrasound volume scanning of a patient anatomy. Examples of an 3D echocardiography probe include, but are not limited to, 3D transesophageal echocardiography (“TEE”) probes and 3D intracardiac echocardiography (“ICE”) probes;

(3) the term “3D transesophageal echocardiography probe” broadly encompasses any and all types of 3D echocardiography probe, as known in the art of the present disclosure or hereinafter conceived, for a ultrasound volume scanning of a patient transesophageal;

(4) the term “3D intracardiac echocardiography probe” broadly encompasses any and all types of 3D echocardiography probe, as known in the art of the present disclosure or hereinafter conceived, for a ultrasound volume scanning of a patient heart;

(5) the term “3D laparoscopic ultrasound probe” broadly encompasses any and all types of 3D echo probes adapted into a 3D laparoscopic ultrasound probe in accordance with the inventive principles of the present disclosure exemplary described herein;

(6) the term “laparoscopic adapter” broadly encompasses any and all types of devices for adapting a 3D transesophageal echocardiography probe into a 3D laparoscopic ultrasound probe in accordance with the inventive principles of the present disclosure exemplary described herein;

(7) the term “laparoscopic sleeve” broadly encompasses any and all types of tubular structures suitable for a laparoscopic procedure in accordance with the inventive principles of the present disclosure exemplary described herein;

(8) the term “robot actuator” broadly encompasses all robot actuators, as known in the art of the present disclosure and hereinafter conceived, for actuation of a deflection of a 3D transesophageal echocardiography probe;

(9) the term “controller” broadly encompasses all structural configurations, as understood in the art of the present disclosure and as exemplary described in the present disclosure, of an application specific main board or an application specific integrated circuit for controlling an application of various inventive principles of the present disclosure as subsequently described in the present disclosure. The structural configuration of the controller may include, but is not limited to, processor(s), computer-usable/computer readable storage medium(s), an operating system, application module(s), peripheral device controller(s), slot(s) and port(s). A controller may be housed within or linked to a workstation. Examples of a “workstation” include, but are not limited to, an assembly of one or more computing devices, a display/monitor, and one or more input devices (e.g., a keyboard, joysticks and mouse) in the form of a standalone computing system, a client computer of a server system, a desktop or a tablet;

(10) the descriptive labels for term “controller” herein facilitates a distinction between controllers as described and claimed herein without specifying or implying any additional limitation to the term “controller”;

(11) the term “application module” broadly encompasses an application incorporated within or accessible by a controller consisting of an electronic circuit and/or an executable program (e.g., executable software stored on non-transitory computer readable medium(s) and/or firmware) for executing a specific application;

(12) the term “position measurement system” broadly encompasses all measurement systems, as known in the art of the present disclosure and hereinafter conceived, for measuring a positon (e.g., a location and/or an orientation) of an object within a coordinate space. Examples of a position measurement system include, but is not limited to, an electromagnetic (“EM”) measurement system (e.g., the Auora® electromagnetic measurement system), an optical-fiber based measurement system (e.g., Fiber-Optic RealShape™ (“FORS”) measurement system), an ultrasound measurement system (e.g., an InSitu or image-based US measurement system), an optical measurement system (e.g., a Polaris optical measurement system), a radio frequency identification measurement system and a magnetic measurement system;

(13) the terms “signal”, “data” and “command” broadly encompasses all forms of a detectable physical quantity or impulse (e.g., voltage, current, or magnetic field strength) as understood in the art of the present disclosure and as exemplary described in the present disclosure for transmitting information and/or instructions in support of applying various inventive principles of the present disclosure as subsequently described in the present disclosure. Signal/data/command communication between various components of the present disclosure may involve any communication method as known in the art of the present disclosure including, but not limited to, signal/data/command transmission/reception over any type of wired or wireless datalink and a reading of signal/data/commands uploaded to a computer-usable/computer readable storage medium; and

(14) the descriptive labels for terms “signal”, “data” and “commands” herein facilitates a distinction between signals/data/commands as described and claimed herein without specifying or implying any additional limitation to the terms “signal”, “data” and “command”.

A first embodiment of the inventions of the present disclosure is a 3D echocardiography probe (e.g., a 3D transesophageal echocardiography probe or a 3D intracardiac echocardiography probe) probe adapted into a 3D laparoscopic ultrasound probe for laparoscopic procedures. The 3D echocardiography probe includes a flexible shaft, and a laparoscopic adapter coupled to the flexible shaft. The laparoscopic adapter adapts the 3D echocardiography probe into the 3D laparoscopic ultrasound probe.

A second embodiment of the inventions of the present disclosure is the laparoscopic adapter employing a laparoscopic sleeve partially encircling or fully encircling (i.e., enclosing) a portion or an entirety of the flexible shaft of the 3D echocardiography probe, and a probe handle mounted to the laparoscopic sleeve.

A third embodiment of the inventions of the present disclosure is a method for adapting the 3D echocardiography probe into the 3D laparoscopic ultrasound probe for laparoscopic procedures. The method involves a coupling of the laparoscopic sleeve to the flexible shaft of the 3D echocardiography probe with the laparoscopic sleeve partially encircling or fully encircling (i.e., enclosing) a portion or an entirety of the flexible shaft. The method further involves a mounting of the probe handle to the laparoscopic sleeve.

A fourth embodiment of the inventions of the present disclosure is a 3D laparoscopic ultrasound system employing a 3D laparoscopic ultrasound probe of the present disclosure, and further employing a robot actuator for actuating a deflection of the flexible shaft of the 3D echocardiography probe and a robot actuator controller for controlling an actuation by the robot actuator of a deflection of the flexible shaft of the 3D echocardiography probe.

A fifth embodiment of the inventions of the present disclosure is a 3D laparoscopic ultrasound system of the present disclosure further employing a probe controller for generating a probe actuation signal indicating a delineated deflecting of the 3D echocardiography probe. The robot actuator controller controls the actuation by the robot actuator of the deflection of the flexible shaft of the 3D echocardiography probe in response to a generation of the probe actuation signal by the probe controller.

A sixth embodiment of the inventions of the present disclosure is a probe handle of the present disclosure including the probe controller for generating a probe actuation signal indicating the delineated deflecting of flexible shaft of the 3D echocardiography probe.

A seventh embodiment of the inventions of the present disclosure is a 3D laparoscopic ultrasound system of the present disclosure further employing a probe measurement system for measuring a position (e.g., a location and/or an orientation) of a volume image by the 3D laparoscopic ultrasound probe within a coordinate system.

An eighth embodiment of the inventions of the present disclosure is a robot actuator controller of the present disclosure incorporates a sensorless force control technique and/or an organ scanning technique.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

FIG. 1A illustrates a first exemplary embodiment of a 3D laparoscopic ultrasound scope in accordance with the inventive principles of the present disclosure.

FIG. 1B illustrates a first exemplary embodiment of a 3D laparoscopic ultrasound actuation system in accordance with the inventive principles of the present disclosure.

FIG. 2A illustrates an exemplary embodiment of a 3D transesophageal echocardiography probe as known in the art of the present disclosure.

FIG. 2B illustrates an exemplary embodiment of a robot actuator for a 3D transesophageal echocardiography probe as known in the art of the present disclosure.

FIGS. 3A-3D illustrates exemplary embodiments of a laparoscopic sleeve in accordance with the inventive principles of the present disclosure.

FIG. 4A illustrates a second exemplary embodiment of a 3D laparoscopic ultrasound scope in accordance with the inventive principles of the present disclosure.

FIG. 4B illustrates a second exemplary embodiment of a 3D laparoscopic ultrasound actuation system in accordance with the inventive principles of the present disclosure.

FIGS. 5A-5E illustrates a third exemplary embodiment of a 3D laparoscopic ultrasound scope in accordance with the inventive principles of the present disclosure.

FIGS. 6A-6C illustrates exemplary embodiments of a 3D laparoscopic ultrasound measurement system in accordance with the inventive principles of the present disclosure.

FIG. 7A illustrates an exemplary embodiment of a robot actuator control scheme in accordance with the inventive principles of the present disclosure.

FIG. 7B illustrates an exemplary embodiment of a robot actuator control scheme incorporating an organ scanning control technique in accordance with the inventive principles of the present disclosure.

FIG. 7C illustrates an exemplary embodiment of a robot actuator control scheme incorporating a sensorless force control technique in accordance with the inventive principles of the present disclosure.

FIG. 7D illustrates an exemplary embodiment of a robot actuator control scheme incorporating a sensorless force control technique and an organ scanning technique in accordance with the inventive principles of the present disclosure.

### DETAILED DESCRIPTION OF THE PREFERRED EMBODIMENTS

The inventions of the present disclosure are premised on an adaptation of a 3D echocardiography probe (e.g., a 3D transesophageal echocardiography (“TEE”) probe or a 3D intracardiac echocardiography (“ICE”) probe) into a 3D laparoscopic ultrasound (“LUS”) probe.

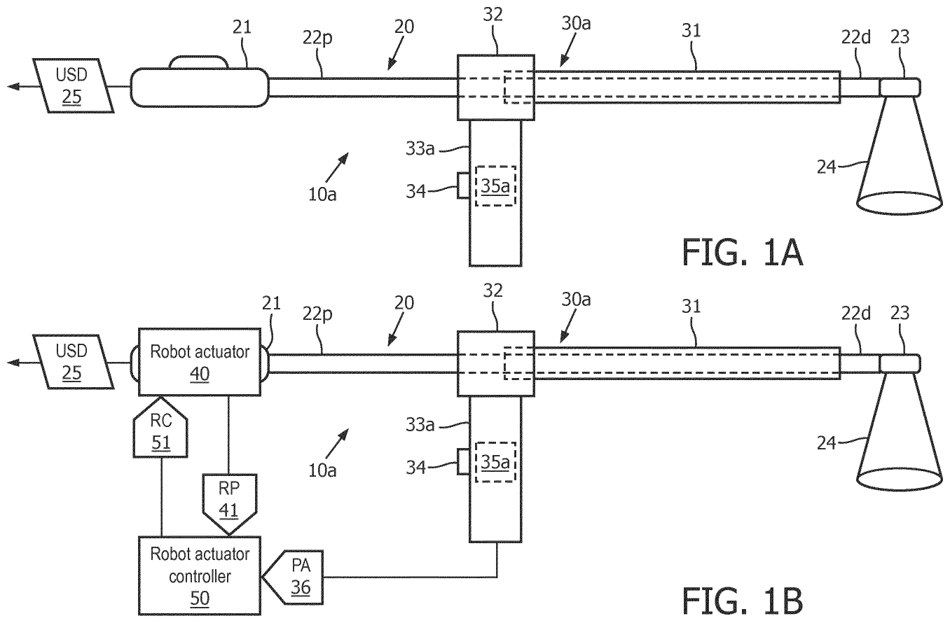

To facilitate an understanding of the inventions of the present disclosure, the following description of FIGS. 1-4 teaches basic inventive principles of an adaptation of a 3D TEE probe into a 3D LUS probe. From the description of FIGS. 1-7, those having ordinary skill in the art will appreciate how to apply the inventive principles of the present disclosure to making and using numerous and varied embodiments of an adaption of a 3D echocardiography probe of any type into a 3D LUS probe of the present disclosure.

Referring to FIG. 1A, an exemplary 3D LUS probe 10a of the present disclosure employs a 3D TEE probe 20 as known in the art of the present disclosure and a laparoscopic adapter 30a of the present disclosure.

3D TEE probe 20 includes an actuation handle 21, a flexible shaft 22 having a proximal end 22p integrated into actuation handle 21 and a 3D ultrasound transducer integrated with a distal end 22d of flexible shaft 22. Actuation handle 21 provides for an actuation of a deflection of distal end 22d of flexible shaft 22 with one (1) or two (2) degrees of freedom as known in the art of the present disclosure. Ultrasound transducer 23 provides for a generation of an ultrasound data 25 representative of an imaging of an ultrasound volume 24 as known in the art of the present disclosure.

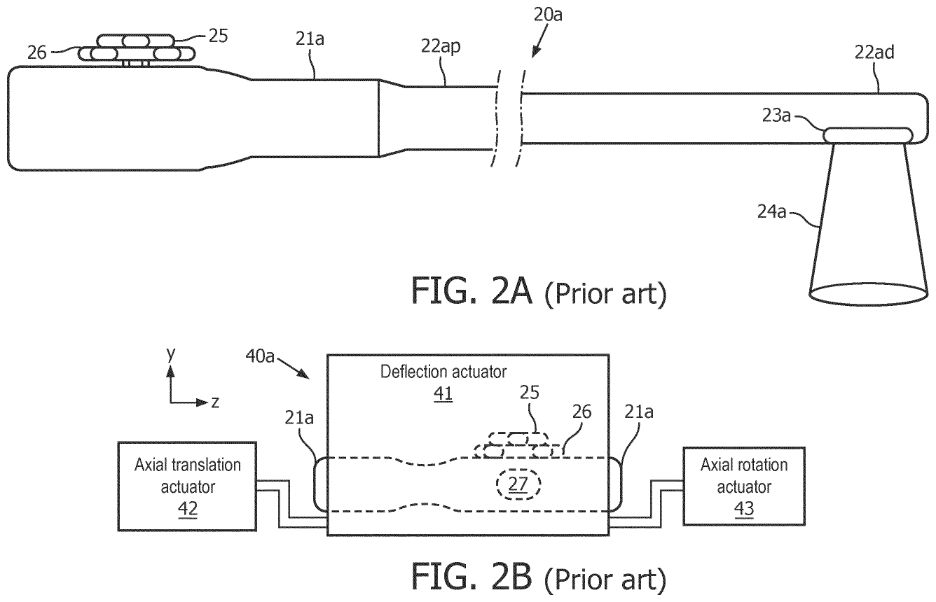

In one embodiment 20a of 3D TEE probe 20 as shown in FIG. 2A, a flexible shaft 22a has a proximal end 22ap integrated into actuation handle 21a and a 3D ultrasound transducer 23a is integrated with a distal end 22d of flexible shaft 22. Actuation handle 21a of 3D TEE probe 20a employs a yaw actuation dial 25 for adjusting a yaw degree freedom of probe head 22ad, and a pitch actuation dial 26 for adjusting a pitch degree freedom of probe head 22ad.

Referring back to FIG. 1A, laparoscopic adapter 30 includes a laparoscopic sleeve 31, an adapter mount 32, and a handle 33a.

Laparoscopic sleeve 31 has a shaft channel for receiving flexible shaft 20 as symbolically shown by the dashed lines within laparoscopic sleeve 31.

In practice, the shaft channel of laparoscopic sleeve 31 may be open whereby laparoscopic sleeve 31 partially encircles a portion of flexible shaft 22 or alternatively, the shaft channel of laparoscopic sleeve 31 may be closed whereby laparoscopic sleeve 31 fully encircles (i.e., encloses) a portion of flexible shaft 22 as shown in FIG. 1A.

Also in practice, the shaft channel laparoscopic sleeve 31 may be dimensioned to loosely or tightly receive flexible shaft therein.

Further in practice, laparoscopic sleeve 31 has a material composition and geometrical configuration suitable for laparoscopic procedures.

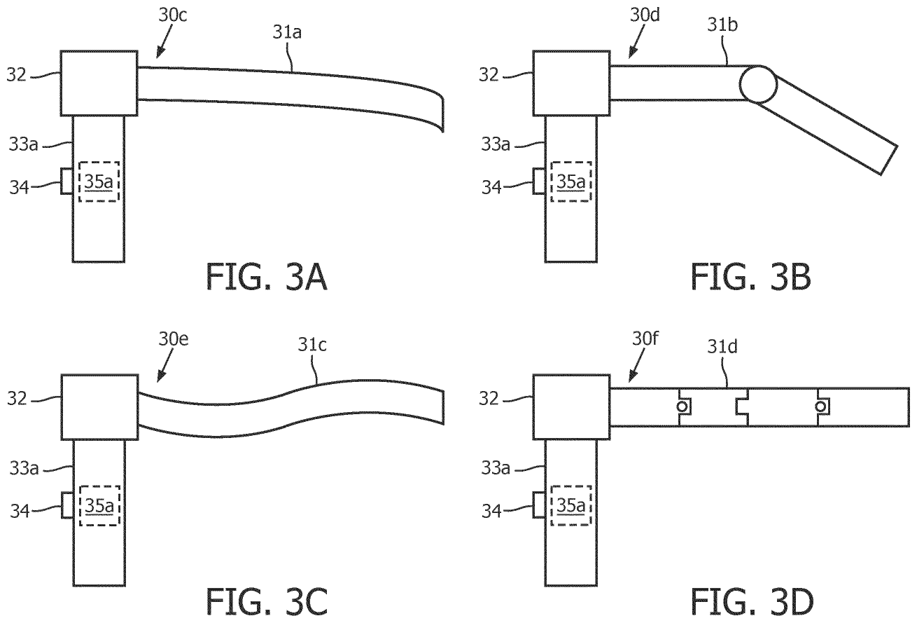

In one embodiment, laparoscopic sleeve 31 may have a rigid material composition with a straight cylindrical configuration as shown in FIG. 1A or a curved cylindrical configuration 31a as shown in FIG. 3A.

In a second embodiment, laparoscopic sleeve 31 may have a rigid material composition with a jointed cylindrical configuration 31b as shown in FIG. 3B for facilitating a manual pivoting of a distal end of laparoscopic sleeve 31.

In a third embodiment, laparoscopic sleeve 31 may have a semi-rigid material composition with a cylindrical configuration, such as, for example, laparoscopic sleeve 31 may be a catheter 31c as shown in FIG. 3C.

In a fourth embodiment, laparoscopic sleeve 31 may have a rigid material composition with a controllable cylindrical configuration, such as, for example, laparoscopic sleeve 31 may be a snake robot 31d as shown in FIG. 3D.

Referring back to FIG. 1A, adapter mount 32 has a mount channel for receiving flexible shaft 20 and/or laparoscopic sleeve 31 as symbolically shown by the dashed lines within laparoscopic sleeve 31.

In practice, adapter mount 32 may clamp onto flexible shaft 20 and/or laparoscopic sleeve 31 to maintain laparoscopic sleeve 31 at a fixed position relative to ultrasound transducer 23.

Still referring to FIG. 1A, probe handle 33a is affixed to adapter mount 32 to enable a user manipulation of a coarse alignment of ultrasound transducer 23 to a region of interest of a patient anatomy.

Probe handle 33a includes a user input device 34 (e.g., a joystick, a rollerball, etc.) for operating a probe controller 35a to generate a probe actuation signal indicating a delineated deflecting of distal end 22d of flexible shaft 22 via user input device 34.

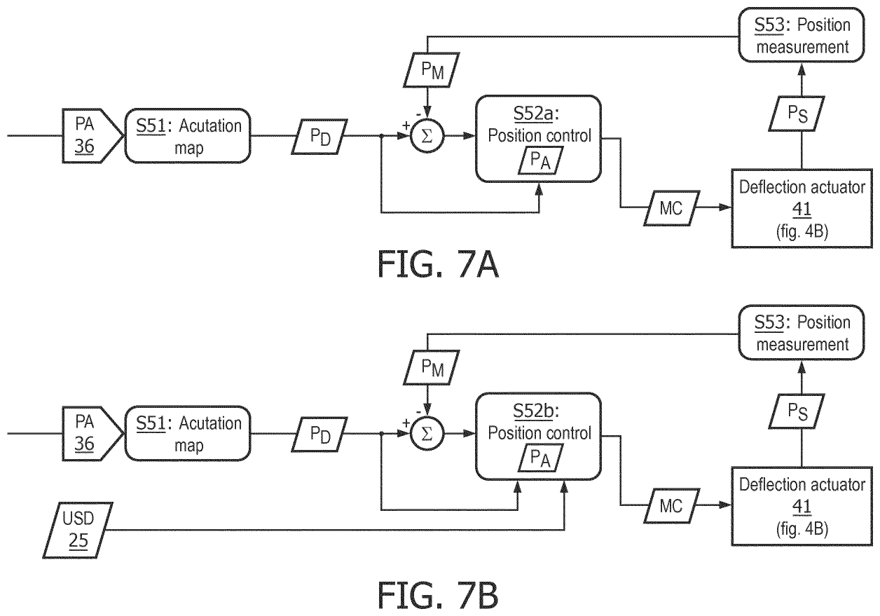

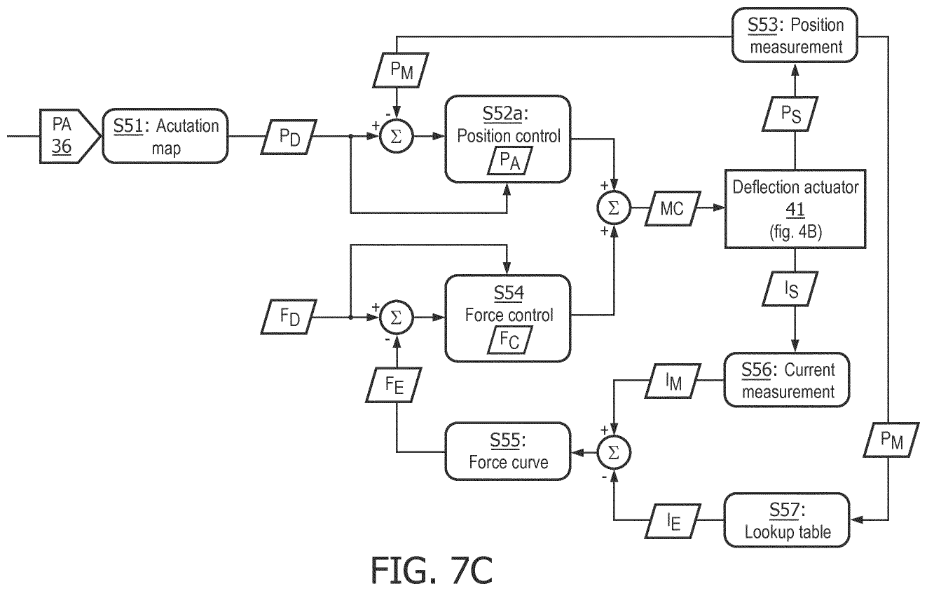

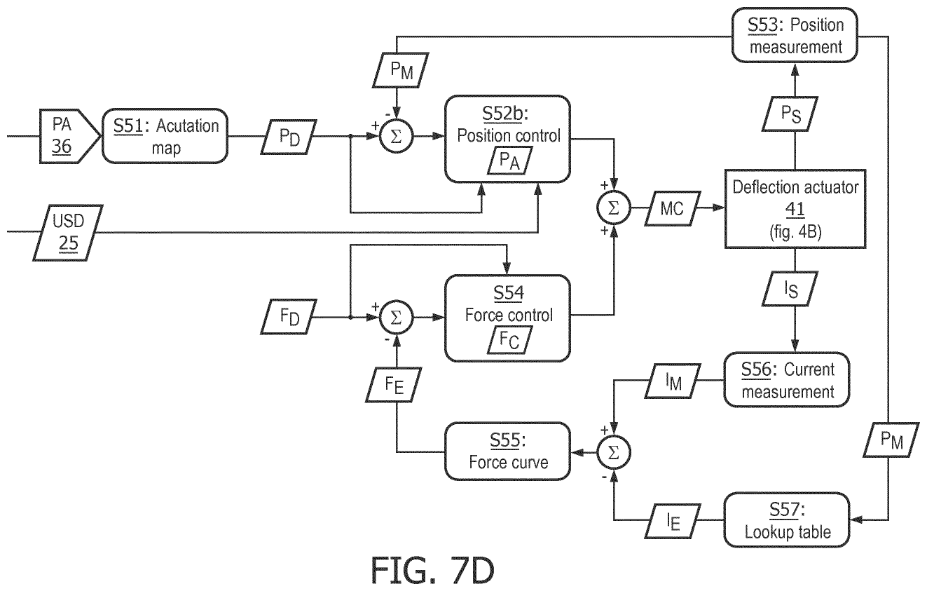

In one embodiment as shown in FIG. 1B, probe controller 35a communicates (wired or wireless) a probe actuation signal 36 to a robot actuator controller 50 whereby robot actuator controller 50 communicates (wired or wireless) robot commands 51 to a robot actuator 40 to control actuation of a deflection of distal end 22d of flexible shaft 22 based on robot position data 41 communicated (wired or wireless) by robot actuator 40 to robot actuator controller 50 as will be further described in connection with FIGS. 7A-7D.

In practice, robot actuator 40 may have any embodiment suitable for actuating a deflection of distal end 22d of flexible shaft 22.

In one embodiment 40a of robot actuator 40 as shown in FIG. 2B, robotic actuator 40a employs a deflection actuator 41, an axial translation actuator 42, and an axial rotation actuator 43 for fine alignment of ultrasound transducer 23a to a region of interest of a patient anatomy.

Deflection actuator 41 is mechanically engaged as known in the art with dials 25 and 26 of TEE probe 20a. Robot actuator controller 50 provides robot commands 51 to motor controller(s) (not shown) of deflection actuator 41 for actuating dials 25 and 26 to actuate a deflection of a TEE probe 20a corresponding to a mapped motion of user input device (e.g., user input device 34 of probe handle 33a).

Axial translation actuator 42 and axial rotation actuator 43 are mechanically coupled to deflection actuator 41.

Axial translation actuator 42 as known in the art may be actuated to translate handle 21a of 3D TEE probe 20a along its longitudinal axis. Robot actuator controller 50 provides further robot commands 51 to a motor controller (not shown) of axial translation actuator 42 to actuate an axial translation of handle 21a, which may correspond to a mapped motion of a user input device (e.g., user input device 34 of probe handle 33a).

Axial rotation actuator 43 as known in the art may be actuated to rotate handle 21a of 3D TEE probe 20a along its longitudinal axis. Robot actuator controller 50 provides further robot commands 51 to a motor controller (not shown) of axial rotation actuator 43 to actuate an axial rotation of handle 21a of 3D TEE probe 20a, which may corresponds to a mapped motion of a user input device (e.g., user input device 34 of probe handle 33a).

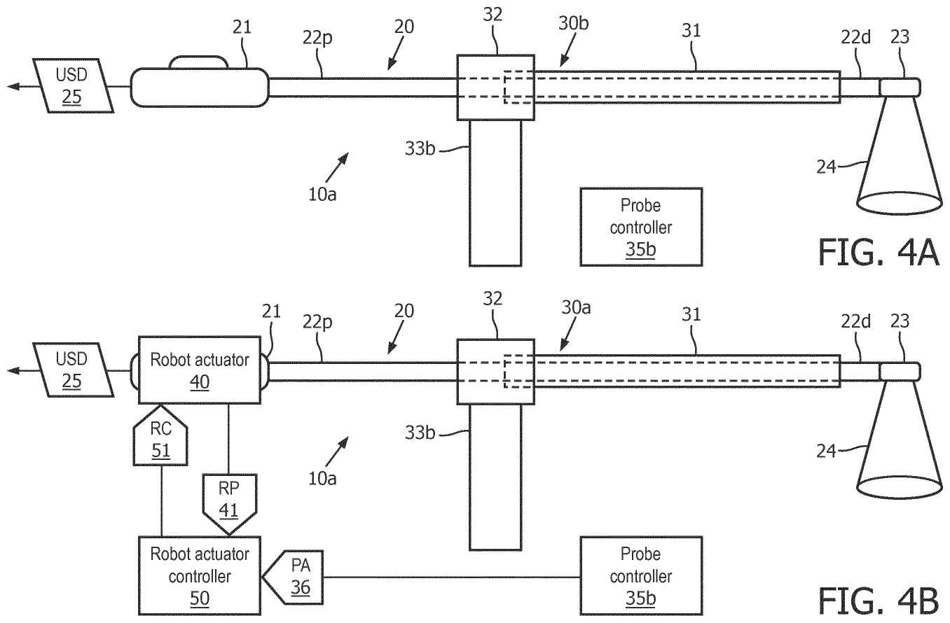

Also in practice, the probe controller may be segregated from the probe handle. For example, as shown in FIGS. 4A and 4B, a probe controller 35b may be segregated from a probe handle 33b whereby probe controller 35b is operated via a user input device integrated with probe handle 33b (e.g., a joystick, a roller ball, etc.) or a user input device segregated from probe handle 33b (e.g., an eye-tracker, a voice control, virtual reality control, gesture tracking, foot pedals, etc.).

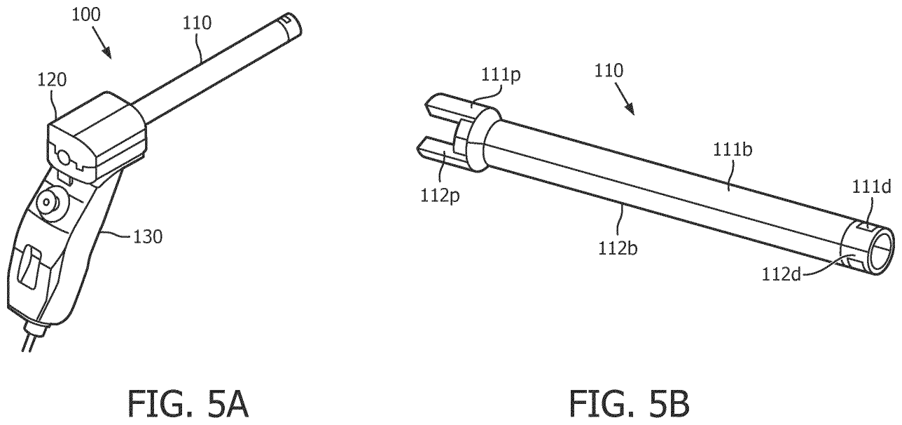

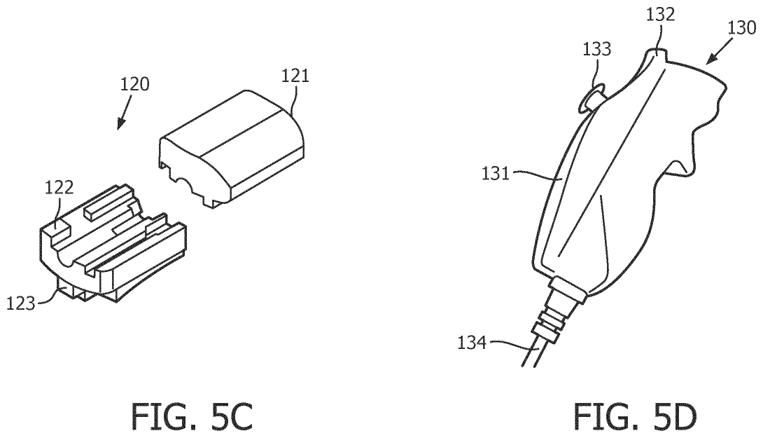

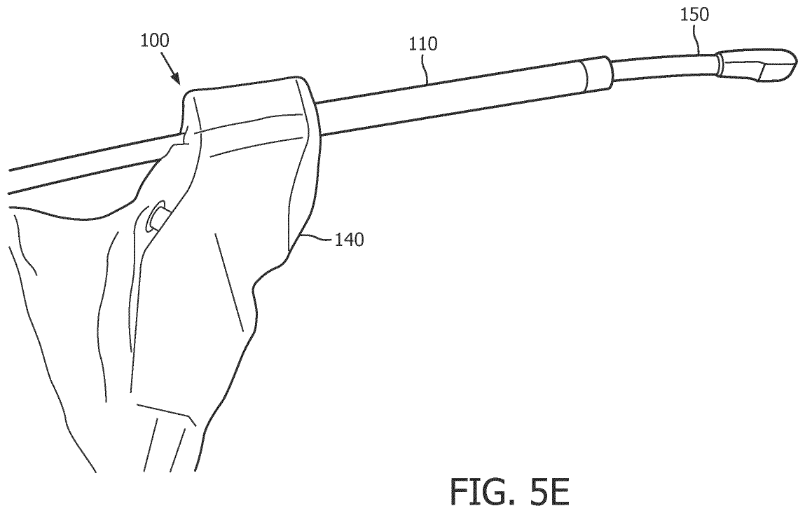

To facilitate a further understanding of the inventions of the present disclosure, the following description of FIGS. 5A-5E teaches a specific embodiment of an adaptation of a 3D transesophageal echocardiography (“TEE”) probe into a 3D laparoscopic ultrasound (“LUS”) probe. From the description of FIGS. 5A-5E, those having ordinary skill in the art will further appreciate how to apply the inventive principles of the present disclosure to making and using numerous and varied embodiments of an adaption of a 3D echocardiography probe of any type into a 3D LUS probe of the present disclosure.

Referring to FIG. 5A, a 3D LUS probe 100 of the present disclosure employs a rigid disposable laparoscopic sleeve 110, a disposable handle mount 120 and a reusable probe handle 130.

As shown in FIG. 5B, laparoscopic sleeve 110 includes an upper semi-cylinder 111 having a shaft cover 111b, a proximal mount coupler 111p and a distal shaft coupler 111d, and a lower semi-cylinder 112 having a shaft cover 112b, a proximal mount coupler 112p and a distal shaft coupler 112d.

As shown in FIG. 5C, handle mount 120 includes an upper mount 121 and a lower mount 122.

As shown in FIG. 5D, probe handle 130 is in the form of a remote probe controller including a handle 131, an extension 132, a joystick 133 and a probe controller (not shown) electrically connected to a cord 134.

Prior to and/or during a laparoscopic procedure, a surgical personal attaches rigid disposable laparoscopic sleeve 110 to a flexible shaft of a US TEE probe by first attaching lower semi-cylinder 112 under the flexible shaft (e.g., distal shaft coupler 112d being a collar) and then, by attaching upper semi-cylinder 111 over the flexible shaft above the lower semi-cylinder 112 (e.g., distal shaft coupler 111d being a flange slidable within a collar). Distal couplers 111d and 112d serve as a clamping mechanism for holding a position of semi-cylinders 111 and 112 on the flexible shaft.

Lower mount 122 is then placed under the flexible shaft and upper mount 122 is slide on lower mount 122 and fixes both the flexible shaft therebetween. Proximal mount couplers 111p and 112p are then friction fitted within upper mount 121 and lower mount 122 (e.g., proximal mount couplers 111p and 112p being flanges friction fitted within slots of upper mount 121 and lower mount 122 respectively).

Reusable remote probe controller 130 is attached to lower controller mount 122, and wrapped with a sterile drape 140 as shown in FIG. 5 (e.g., lower controller mount 122 including a rail 123 slidable within a groove (not shown) of controller 130 to a stop 132).

Upon adaption of the 3D TEE probe into a 3D LUS probe, the handle of 3D LUS probe is inserted into an actuator robot that controls its dials. Both a direction and a speed of a deflection of the 3D LUS probe are controlled by reusable remote probe controller 130.

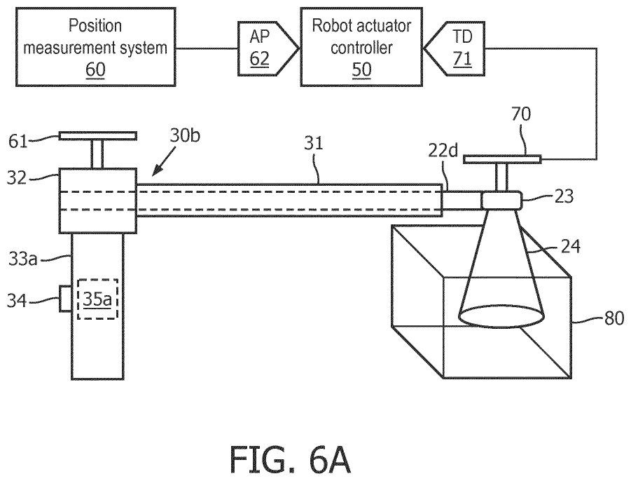

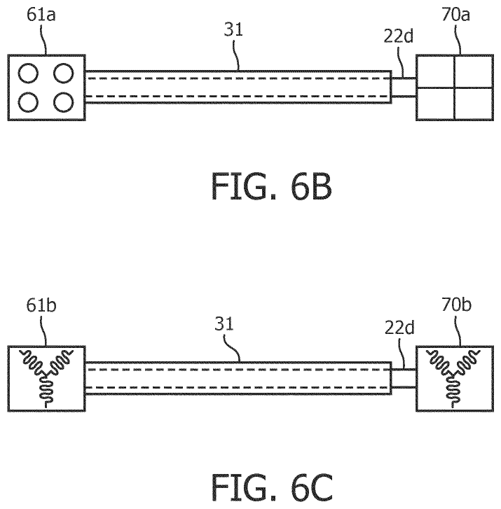

To facilitate a further understanding of the inventions of the present disclosure, the following description of FIGS. 6 and 7 teaches various deflection control schemes executed by a robot actuator controller of the present disclosure. From the description of FIGS. 5A-5E, those having ordinary skill in the art will further appreciate how to apply the inventive principles of the present disclosure to making and using numerous and varied embodiments of deflection control schemes of the present disclosure.

Referring to FIG. 6A, a laparoscopic adapter 31 of the present disclosure is tracked by an external position measurement system (PMS) 60 for generation an adapter pose signal estimating a 3D pose of laparoscopic adapter 31 using a tracked body 61 rigidly attached to handle mount 32 and pre-calibrated so that a position of laparoscopic adapter 31 with respect to PSM 60 in known. A registration between a pre-operative image volume 80 (e.g., a CT volume or a MRI volume) and 3D US volume 25 is provided by PSM 60 using registration techniques known in art of the present disclosure including, but not limited to, a point-based rigid registration based on anatomical landmarks (vessel's bifurcations, calcifications, etc.).

A registration between 3D US volume 24 and tracked body 61 rigidly attached to handle mount 32 is calculated using registration techniques known in art of the present disclosure including, but not limited to, a registration technique utilizing a calibration tool made of ultrasound opaque features organized at a known geometry (hereinafter the “opaque registration”). An exemplary calibration tool 61a is shown in FIG. 6B.

For the opaque registration, ultrasound transducer 23 of 3D LUS probe must be stationary with respect to handle mount 32. An orientation of 3D US transducer 23 is therefore tracked using an inertial measurement unit (IMU) 70 as known in art of the present disclosure including, but not limited to, a combination 70a of an accelerometer, a gyroscope and a magnetometer as shown in FIG. 6B. IMU 70 generates a transducer orientation signal 71 indicative any deflection of 3D US transducer 23.

Robot actuator controller 50 processes adapter position signal 62 and transducer orientation signal 71 to control an actuation of a deflection of 3D US transducer 23. Assuming a link between the tracked body 61 rigidly attached to handle mount 32 and 3D US transducer 23 is not compressible, a position (i.e., a location and/or an orientation) of the US volume 24 with respect to pre-operative image volume 80 may be estimated using one of the mathematical models known in art of the present disclosure for single-link flexible manipulator based transducer orientation signal 71.

Alternatively, as shown in FIG. 6C, an electromagnetic sensor array 61b may be rigidly attached to handle mount 32 and an electromagnetic sensor array 70b may be attached to 3D US transducer 23.

Referring to FIG. 7A, a robot actuator control scheme executable by robot actuator controller 50 (FIGS. 1B and 4B). Basically, a stage S41 involves a generation a desired actuation position P_{D} of 3D US transducer 23 (FIGS. 1B and 4B) derived from a mapping by robot actuator controller 50 of probe actuation signal PAS as previously described herein, and a position control stage S52a generates an actuation position P_{A} for 3D US transducer 23 in terms of a specific pitch and yaw achieved by corresponding angular positions of the gears/actuation dials.

A generation of motor commands MC for achieving actuation position P_{A} involves a minimization a position error between actuation position P_{A} and measured motor positions P_{M}.

Specifically, a motor controller of deflection actuator 41 (FIG. 41) continually communicates sensed motor positions P_{S} during a stage S53 to robot actuator controller 50. In response thereto, robot actuator controller 50 periodically measures sensed motor positions P_{S} and compares the measured motor positions P_{M} to motor positions associated with the desired actuation position P_{D} of 3D US transducer 23 and the resulting position error is an input for position control stage S52a designed to minimize the position error. In practice, robot actuator controller 50 may execute any control technique(s) as known in the art of the present disclosure for minimizing the position error (e.g., a PID control).

Robot actuator controller 50 continually loop through stages S51-S53 during the laparoscopic procedure.

Referring to FIG. 7B, the robot actuator control scheme of FIG. 7A involves a input of a tracked US volume 25 for purposes of implementing an organ scanning technique. Specifically, robot actuator controller 50 is able to ascertain a spatial relation between 3D US transducer 23 and a surface of an organ whereby robot actuator controller 50 constrains a movement of 3D US transducer 23 as needed to maintain a parallel spatial relation between 3D US transducer 23 and the surface of an organ while minimizing a position error.

Referring to FIG. 7C, the robot actuator control scheme of FIG. 7A involves an implementation of a sensorless force control technique.

Specifically, a desired force F_{D} of 3D US transducer 23, which is typically a constant value greater than zero to maintain contact with tissue and ensure acoustic coupling, is communicated to robot actuator controller 50 during a force control stage S54 whereby robot actuator controller 50 generates a contact force correction F_{C} for actuation position P_{A} for 3D US transducer 23.

The generation of motor commands MC involves an application of contact force correction F_{C} to actuation position P_{A} in view of minimizing a position error between actuation position PA and measured motor positions P_{M}, and a contract force error between contact force correction F_{C} and an expected contact force F_{E}.

Specifically, a motor controller of deflection actuator 41 continually communicates sensed motor positions P_{S} and sensed motor currents I_{S} during respective stages S53 and S56 to robot actuator controller 50. In response thereto, robot actuator controller 50 periodically measures sensed motor positions P_{S} and compares the measured motor positions P_{M} to motor positions associated with a desired actuation position P_{D} of 3D US transducer 23 and the resulting position error is an input for position control stage S52a designed to minimize the position error. In practice, robot actuator controller 50 may execute any control technique(s) as known in the art for minimizing the position error (e.g., a PID control).

Robot actuator controller 50 also periodically in sync measures sensed motor currents I_{S} and combines the measured sensed motor currents I_{S} to an expected motor currents I_{E}, which is calculated by inputting measured motor positions P_{M} into the lookup table of stage S57 computed by a calibrator as known in the art of the present disclosure. The lookup table takes two inputs of position of the two dials and returns two expected current values I_{E} for each degree-of-freedom. During stage S55 expected current values I_{E} and the measured motor current values I_{M} are current fed to force curve (C→F) computed by calibrator as known in the art of the present disclosure to estimate an expected contact force F_{E} on 3D US transducer 23.

Force control stage S54 receives contact force correction F_{C} from a comparison of desired contact force F_{D} and expected contract force F_{E} and adjusts a path generated by position control stage S52 to limit the forces exerted by the head of 3D US transducer 23. In one embodiment, a direct method to model this motion is to assume that contact surface acts as an ideal spring, in which case:

Δf=K(x−xo)

where Δf is the force error signal, x is the position of the contact point, xo would be the position of 3D US transducer 23 if there was no obstacle, and K is elastic constant of the esophagus of the patient (values known in literature can be used). Since x₀ can be known from the kinematic model of 3D US transducer 23, there is a direct link between motor commands and the force. Similarly to position control value:

$$x = \frac{Δ f}{K} + x 0$$

Robot actuator controller 50 continually loop through stages S51-S57 during the laparoscopic procedure.

Referring to FIG. 7D, the robot actuator control scheme of FIG. 7C involves a input of a tracked US volume 25 for purposes of implementing an organ scanning technique. As previously described, robot actuator controller 50 is able to ascertain a spatial relation between 3D US transducer 23 and a surface of an organ whereby robot actuator controller 50 constrains a movement of 3D US transducer 23 as needed to maintain a parallel spatial relation between 3D US transducer 23 and the surface of an organ while minimizing a position error.

Referring to FIGS. 1-7, those having ordinary skill in the art of the present disclosure will appreciate numerous benefits of the inventions of the present disclosure including, but not limited to, an acquisition of volumetric information during a laparoscopic procedure.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk-read only memory (CD-ROM), compact disk-read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive adaptions of a 3D echocardiography probe of any type (e.g., a 3D TEE probe or a 3D ICE probe) into a 3D LUS probe of the present disclosure (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A laparoscopic adapter for adapting a 3D echocardiography probe into a 3D laparoscopic ultrasound probe, the laparoscopic adapter comprising:
a laparoscopic sleeve configured to at least partially encircle a portion of a flexible shaft of the 3D echocardiography probe; and
a probe handle mountable to the laparoscopic sleeve.

2. The laparoscopic adapter of claim 1,
wherein the laparoscopic sleeve includes a first semi-cylinder affixable to a second semi-cylinder; and
wherein the first semi-cylinder and the second semi-cylinder are configured to enclose the portion of the flexible shaft.

3. The laparoscopic adapter of claim 1,
wherein the laparoscopic sleeve is one of a rigid cylinder, a jointed cylinder, a catheter and a snake robot.

4. The laparoscopic adapter of claim 1, further comprising:
a handle mount for mounting the probe handle to the laparoscopic sleeve, wherein the handle mount is configured to at least partially encircle a portion of the laparoscopic sleeve.

5. The laparoscopic adapter of claim 4,
wherein the handle mount includes a first mount affixable to a second mount;
wherein the first mount and the second mount are configured to enclose the portion of the laparoscopic sleeve; and
wherein the probe handle is configured to be affixed to at least one the first mount and the second mount.

6. The laparoscopic adapter of claim 1,
wherein the probe handle includes a probe controller configured to generate a probe actuation signal indicating a delineated deflecting of the flexible shaft.

7. The laparoscopic adapter of claim 6,
wherein the probe handle further includes a user input device configured to operate the probe controller.

8. A 3D laparoscopic ultrasound probe for laparoscopic procedures, the 3D laparoscopic ultrasound probe comprising:
a 3D echocardiography probe including a flexible shaft; and
a laparoscopic adapter coupled to the flexible shaft,
wherein the laparoscopic adapter adapts the 3D echocardiography probe into the 3D laparoscopic ultrasound probe.

9. The 3D laparoscopic ultrasound probe of claim 8, wherein the laparoscopic adapter includes a laparoscopic sleeve at least partially encircling a portion of the flexible shaft.

10. The 3D laparoscopic ultrasound probe of claim 9,
wherein the laparoscopic sleeve includes a first semi-cylinder affixed to a second semi-cylinder; and
wherein the first semi-cylinder and the second semi-cylinder enclose the portion of the flexible shaft.

11. The 3D laparoscopic ultrasound probe of claim 9,
wherein the laparoscopic sleeve is one of a rigid cylinder, a jointed cylinder, a catheter and a snake robot.

12. The 3D laparoscopic ultrasound probe of claim 9,
wherein the laparoscopic adapter further includes a probe handle and a handle mount for mounting the probe handle to the laparoscopic sleeve; and
wherein the handle mount at least partially encircling a portion of the laparoscopic sleeve.

13. The 3D laparoscopic ultrasound probe of claim 12,
wherein the handle mount includes a first mount affixed to a second mount;
wherein the first mount and the second mount enclose the portion of the laparoscopic sleeve; and
wherein the probe handle is configured to be affixed to at least one the first mount and the second mount.

14. The 3D laparoscopic ultrasound probe of claim 12,
wherein the probe handle includes a probe controller configured to generate a probe actuation signal indicating a delineated deflecting of the flexible shaft.

15. The 3D laparoscopic ultrasound probe of claim 4,
wherein the probe handle further includes a user input device configured to operate the probe controller.

16. A method for adapting a 3D echocardiography probe into a 3D laparoscopic ultrasound probe for laparoscopic procedures, the method comprising:
coupling a laparoscopic sleeve to a flexible shaft of the 3D echocardiography probe,
wherein the laparoscopic sleeve at least partially encircles a portion of the flexible shaft; and
mounting a probe handle to the laparoscopic sleeve.

17. The method of claim 16,
wherein the laparoscopic sleeve includes a first semi-cylinder and a second semi-cylinder; and
wherein the coupling of the laparoscopic sleeve to the flexible shaft of the 3D echocardiography probe includes:
positioning the portion of the flexible shaft within the first semi-cylinder;
affixing the second semi-cylinder onto the first semi-cylinder.

18. The method of claim 16, wherein the mounting of the probe handle to the laparoscopic sleeve includes:
affixing a handle mount to the laparoscopic sleeve,
wherein the handle mount partially encircles a portion of the laparoscopic sleeve; and
affixing the probe handle to the handle mount.

19. The method of claim 18, wherein the affixing of the handle mount to the laparoscopic sleeve includes:
positioning the portion of the laparoscopic sleeve within a first mount; and
affixing a second mount to the first mount.

20. The method of claim 16, further comprising:
draping the probe handle.
